(19) United States
(12) Patent Application Publication
(10) Pub. No.: US 2006/0135889 A1
(43) Pub. Date: Jun. 22, 2006

(54) Fitness device

(76) Inventor: Wendelin Egli, Seuzach (CH)

Correspondence Address: JENKENS & GILCHRIST, PC, 1445 ROSS AVENUE, SUITE 3200, DALLAS, TX, 75202, US

(21) Appl. No.: 10/529,335

(22) PCT Filed: May 1, 2003
(86) PCT No.: PCT/CH03/00283

§ 371 Date: Nov. 21, 2005

(30) Foreign Application Priority Data
Sep. 25, 2002 (CH) 1610/02

(51) Int. Cl. A61H 7/00; A41D 1/06; A61B 5/02; A61B 5/08; A61B 5/05; A61B 5/00

(52) U.S. Cl. 601/11; 2/227; 600/549; 600/547; 600/500; 600/529

(57) ABSTRACT

The inventive fitness device is embodied in the form of an article of clothing, consisting of a pair of trousers, for example, consisting of an airtight outer skin (1) made of an elastic plastic material, and a distancing layer (2) which is arranged thereunder and made of a distancing knitted fabric consisting of monolfilament fibres. Sealing edges (6) are produced by wrapping the outer skin (1) over the distancing layer (2). A vacuum tube (4) can be connected to a connection piece (3). A portable vacuum pump (5) produces a medically and advantageously indexed negative differential pressure in relation to atmospheric pressure, which extends into the entire article of clothing as a result of the distancing layer (2). The vacuum pump (5) is fastened to a strip (7) which can be worn on the shoulders or in an otherwise appropriate position.

The present invention pertains to a fitness device in the form of an article of clothing according to the preamble of claim 1.

Fitness devices of this type are known—under various designations—from U.S. Pat. No. 726,791 (D1), U.S. Pat. No. 4,230,114 (D2) and WO 01/52787 (D3). The present invention and the related state of the art are based on the notion that the blood circulation, particularly of the subcutaneous fat tissue—that usually has an inferior blood circulation, can be improved by subjecting certain sections of the human body to a slight external vacuum. If the metabolic rate is simultaneously increased by means of physical activity, more fat tissue can be metabolized due to the improved blood circulation. For example, the vacuum can be applied by arranging a stationary training device, e.g., a bicycle ergometer or a treadmill, in a vacuum chamber, wherein persons interested in this type of training under vacuum conditions enter the vacuum chamber that typically accommodates the body to a height above the hips. An elastic and airtight seal adjoins the body above the hips. Training devices of this type are known, for example, from U.S. Pat. No. 5,133,339 and EP 0 906 774. However, such stationary systems are quite costly and the users need to frequent the facilities, in which these systems are installed. In addition, the athletic or, in more general terms, physical activity is extremely limited. These disadvantages were at least partially eliminated with the devices described in D1-D3.

The main disadvantage of an article of clothing that should serve as a replacement for a stationary system of the above-described type can be seen in the fact that such an article of clothing needs to contain an airtight outer skin. When a negative differential pressure referred to the atmospheric pressure is generated within this outer skin by means of a stationary (D1, D2) or portable (D3) air pump, the atmospheric pressure acts upon the entire partially evacuated surface of this article of clothing such that it is pressed against the body surface. This decisively impairs the evacuation of the space between the article of clothing and the body surface.

This is taken into account with the spacers claimed in D1 and D3. These spacers are realized rigidly in D1 and consist of pressurized tubular pneumatic elements in D3. However, these spacers act as reinforcing ribs together with the outer skin that is subjected to the differential pressure. Such reinforcing ribs impair the athletic or, in more general terms, physical activity of the wearer. Despite these known spacers, it is still possible for the outer skin to locally adjoin the body surface such that the function of this article of clothing, namely an improvement in the blood circulation of the subcutaneous fat tissue, is not achieved at all locations.

The present invention aims to disclose a spacer that eliminates the aforementioned disadvantages and makes it possible to simplify the overall design of the article of clothing and the corresponding pump unit while simultaneously lowering the costs substantially.

This objective is attained with the characteristics disclosed in the characterizing portion of claim 1, wherein other advantageous embodiments of the invention are disclosed in the dependent claims.

The invention is described in greater detail below with reference to the enclosed figures. The figures show:

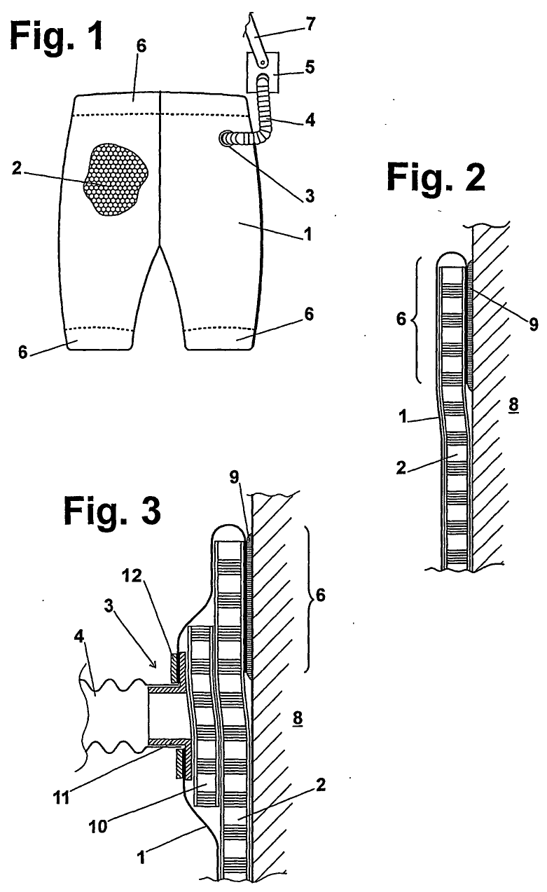

FIG. 1, a top view of one embodiment of the invention;

FIG. 2, a longitudinal section through a part of FIG. 1, and

FIG. 3, a longitudinal section through a special part of FIG. 1.

FIG. 1 shows a top view of an article of clothing according to the invention that is realized in the form of shorts. These shorts comprise an outer skin 1 that is made of an airtight and elastic material, for example, polyurethane (PU). A spacing layer 2 that is essentially cut into the same shape as the outer skin 1 lies underneath this outer skin 1. The spacing layer consists of a knitted spacer fabric. Knitted spacer fabrics are basically known in the textile industry and need to consist of monofilament fibers if the desired purpose should be fulfilled. Naturally, it would also be possible to utilize knitted spacer fabrics consisting of different textile fibers; the decisive factor in this respect is superior air permeability in the directions perpendicular to the outer skin 1.

A connection piece 3 for a vacuum hose 4 is arranged at a suitable location of the outer skin 1. This vacuum hose leads to a portable, battery-operated vacuum pump that is preferably accommodated in a pouch 5 with a strap 7. The aforementioned vacuum pump is rated such that it can generate and maintain a differential pressure between 10 and 50 hPa referred to the atmospheric pressure at the expected leakage rate of the sealing edges 6. The strap 7 is designed for wearing the pump over the shoulder or for attaching the pump to the body in another suitable fashion. Articles of clothing with a complex shape or for ensuring a uniform vacuum over the entire body surface in question naturally may also comprise two or more connection pieces 3, as well as a corresponding number of vacuum hoses 4 or, if so required, one branched vacuum hose 4. FIG. 2 shows a longitudinal section through the upper part of one side of the article of clothing according to FIG. 1. The individual parts are illustrated slightly spaced apart from one another in order to provide a better overview. In the region of the sealing edge 6, the outer skin 1 consisting of an elastic plastic material, for example, is folded inward over the spacing layer 2 and directly adjoins the body identified by the reference symbol 8. If so required, a thin layer 9 of a suitable cream or gel may be applied between the inwardly folded part of the outer skin 1 and the body surface.

The outer skin 1 and the spacing layer 2 are respectively cut ready-to-wear into the respective shape and size of the article of clothing such that they can be washed separately. Since the two parts 1, 2 are connected by simply folding the outer skin 1 inward, no connecting elements need to be disengaged in order to clean the article of clothing. If a suitable cut is chosen for the spacing layer 2, it can be ensured that only a small selection of ready-to-wear sizes is required.

FIG. 3 shows a longitudinal section through the article of clothing in the region of the connection piece 3. In order to improve the air throughput perpendicular to the spacing layer 2, a second layer 10 of the knitted spacer fabric can be applied onto the spacing layer 2 at this location and connected thereto. The connection piece 3 may be designed in such a way that a hat-shaped part 11 thereof extends through a preferably round opening in the outer skin 1, wherein a clamp 12 is placed over the cylindrical section of the part 11 such that it presses the edge of the round opening in the outer skin 1 against the flat section of the part 11. The vacuum hose 4 is subsequently pushed onto the aforementioned cylindrical section of the part 11 and fixed thereon. Other suitable connections are generally known and customarily used in the field of connecting systems.

The spacing layer 2 provided easily makes it possible to arrange sensors for monitoring certain functions of the person wearing the article of clothing. Such sensors may be provided, for example, for measuring the skin temperature at different locations, the conductivity of the skin, the pulse or the breathing rate, wherein the latter can be determined, for example, based on the tension in the abdominal region of the article of clothing. These sensors may be connected to a suitable data collecting device that is also accommodated in a pouch 7 and supplied with power by the same battery as the vacuum pump according to the invention. This indicates that the fitness device in the form of an article of clothing can also be utilized as a measuring and monitoring device in the field of sports medicine, namely because the athletic activity—particularly with respect to endurance sports—is hardly restricted by the article of clothing. In addition, costly and restricting telemetry devices can be eliminated by providing a suitable data collecting device.

Although the shorts shown in FIG. 1 presumably represent the most popular article of clothing according to the invention, it should be understood that this embodiment merely has an exemplary character. According to the invention, it would also be possible to realize ankle-length trousers, a top that only subjects parts of the upper body—for example, the upper arms—to a partial vacuum or even a full-body garment, all of which comprise the airtight outer skin 1 and the spacing layer 2. In all variations of the invention, it is possible to exclude certain sections of the body from the partial vacuum, in which case these sections are simply covered by a suitable air-permeable textile material.

1. A fitness device in the form of an article of clothing, comprising an airtight and elastic outer skin (1), a vacuum pump for generating a partial vacuum between the body surface of the person wearing the article of clothing and the outer skin (1), and a spacer between the body surface and the outer skin (1), wherein the vacuum pump is realized in a battery-operated fashion and can be carried along in a pouch 15, and wherein the vacuum pump is connected to a connection piece (3) arranged on the article of clothing by means of a vacuum hose (4),
characterized in that the spacer consists of a textile spacing layer (2) that is at least partially cut into the same shape as the article of clothing.

2. The fitness device according to claim 1, characterized in that the spacing layer (2) consists of a knitted spacer fabric that is made of monofilament fibers.

3. The fitness device according to claim 2, characterized in that the article of clothing contains sealing edges (6) that are formed by the elastic outer skin (1).

4. The fitness device according to claim 3, characterized in that the sealing edges consist of sections that are folded over the spacing layer (2).

5. The fitness device according to claim 2, characterized in that the article of clothing covers the entire body between the neck and the ankles.

6. The fitness device according to claim 2, characterized in that the article of clothing consists of trousers that extend above the hips.

7. The fitness device according to claim 6, characterized in that the trousers are realized in the form of ankle-length trousers.

8. The fitness device according to claim 6, characterized in that the trousers end underneath the thighs.

9. The fitness device according to claim 2, characterized in that the article of clothing at least partially covers the upper body only.

10. The fitness device according to claim 2, characterized in that
the spacing layer (2) is cut ready-to-wear into the respective shape and size of the article of clothing, and in that
the outer skin (1) and the spacing layer (2) are not connected to one another.

11. The fitness device according to claim 2, characterized in that
the article of clothing contains sensors for acquiring predetermined body parameters of the person wearing the article of clothing, in that
a data collecting device is provided and connected to the aforementioned sensors, and in that
said data collecting device is portable.

12. The fitness device according to claim 11, characterized in that it comprises at least one skin temperature sensor.

13. The fitness device according to claim 11, characterized in that it comprises at least one skin conductivity sensor.

14. The fitness device according to claim 11, characterized in that it comprises a pulse frequency sensor.

15. The fitness device according to claim 11, characterized in that it comprises a breathing rate sensor.

16. The fitness device according to claim 1, characterized in that it comprises more than one connection piece (3) and a corresponding number of vacuum hoses (4).
